(19) United States
(12) Patent Application Publication
(10) Pub. No.: US 2007/0185560 A1
(43) Pub. Date: Aug. 9, 2007

(54) Expandable stent

(75) Inventors: Blayne A. Roeder, Lafayette, IN (US); Richard A. Swift, South Bend, IN (US); David D. Grewe, West Lafayette, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/604,573

(22) Filed: Nov. 27, 2006

Related U.S. Application Data
(60) Provisional application No. 60/740,005, filed on Nov. 28, 2005.

(51) Int. Cl. A61F 2/06

(52) U.S. Cl. 623/1.15; 623/1.16

(57) ABSTRACT

An expandable stent for medical implantation is described which has a generally cylindrical structure with a central longitudinal axis. At least a portion of the generally cylindrical structure is formed from an arrangement of circumferentially curved structural members. The portion expands from a compressed state to an expanded state by a spiraling motion of each of the structural members about the central longitudinal axis. In the compressed state, each of the structural members may nest within an open region formed by segments composing the structural member, such that the portion of the generally cylindrical structure has no overlapping regions. A ratio of a circumferential length of each structural member to a spacing between adjacent structural members may be in the range of about 1.8 to about 2.3.

### EXPANDABLE STENT

The present patent document claims the benefit of the filing date under 35 U.S.C. §119(e) of Provisional U.S. patent application Ser. No. 60/740,005, filed Nov. 28, 2005, which is hereby incorporated by reference.

### TECHNICAL FIELD

The present invention relates generally to medical devices and more particularly to expandable stents.

### BACKGROUND

Stents are useful in a variety of medical procedures and are often used to treat blockages, occlusions, narrowing ailments and other related problems that restrict flow through a passageway. Stents are typically designed as tubular support structures that are implanted within an artery or other vessel and then expanded from a compressed diameter to an expanded diameter. Once the stent is positioned and expanded at the area to be treated, the tubular support structure of the stent contacts and radially supports the inner wall of the passageway, thereby preventing it from closing. Stents are generally classified as either balloon-expandable or self-expandable. Balloon-expandable stents expand in response to the inflation of a balloon. Self-expandable stents, on the other hand, expand automatically when released from a delivery device.

Numerous vessels throughout the vascular system, including peripheral arteries, such as the carotid, brachial, renal, iliac and femoral arteries, and other vessels, may benefit from treatment by a stent. For example, the superficial femoral artery (SFA) may be a site of occlusions or blockages caused by peripheral artery disease. This condition causes leg pain and gangrene in severe cases and affects roughly 8 million to 12 million Americans according to the American Heart Association.

One challenge of designing a stent to treat the SFA is that peripheral arteries are particularly susceptible to external traumas. Such traumas could damage an implanted stent, impairing its ability to hold the vessel open. Conventional stents formed from, for example, 304 stainless steel tend to deform plastically (i.e., nonrecoverably) in response to applied stresses and thus may be permanently crushed by the force of such traumas. In order to successfully treat occlusions in the SFA and other peripheral arteries, a stent preferably would be able to deform elastically (i.e., recoverably) in response to external stresses and revert back to its original expanded shape when the stress is relieved.

Preferably, the stent is designed in a configuration that allows a large and uniform radial force to be exerted on the vessel wall when the stent is deployed. This is preferred to ensure that the stent, in its expanded state, compresses occlusions and holds the vessel open. A stent that exerts a large and uniform radial force when deployed is also better able to resist external traumas.

Additionally, it is desirable that the stent be highly flexible axially both in bending and torsion to accommodate the stresses that are experienced by the implanted stent as a result of body motions and the curvature inherent to body vessels.

Good fatigue properties are also preferred since the stent may be exposed to multiple external traumas while implanted. With each trauma, the stent must withstand the impact and return to its original configuration. In vessels such as the SFA, a stent is also subject to constant expansion and compression due to the pulsation of blood flow, which also must be considered in designing the fatigue behavior of a stent.

Finally, a uniform circumferential stiffness is desirable to minimize the difficulties associated with compressing or crimping the stent to a reduced diameter for delivery into a vessel for treatment.

Current stent designs do not satisfy these requirements or provide these advantages.

### BRIEF SUMMARY

A stent for medical implantation is described that may overcome the limitations of current stent designs. The stent may be particularly useful in the treatment of peripheral arteries, such as the SFA. The stent is designed to provide a substantial amount of elastic (i.e., recoverable) deformation in response to an external trauma, a high and uniform radial force when deployed, high axial flexibility in bending and torsion, a long fatigue life, and a uniform circumferential stiffness.

According to one embodiment, the stent has a generally cylindrical structure with a central longitudinal axis, and at least a portion of the generally cylindrical structure is formed from an arrangement of circumferentially curved structural members. Each structural member is formed from two segments, each segment having a first end and a second end. The first end of one segment connects to the first end of the other segment, thereby forming the structural member. The second end of each segment connects to another segment of a different structural member, such that the structural members are oppositely and alternately positioned in a longitudinal direction, thereby forming the portion of the generally cylindrical structure. The portion expands from a compressed state to an expanded state by a spiraling motion of each of the structural members about the central longitudinal axis. In the compressed state, each of the structural members nests within an open region formed by the segments such that the portion of the generally cylindrical structure has no overlapping regions. A ratio of a circumferential length of each structural member to a spacing between adjacent structural members is in the range of from about 1.8 to about 3.2.

According to another embodiment, the stent has a generally cylindrical structure with a central longitudinal axis, and at least a portion of the generally cylindrical structure is formed from an arrangement of circumferentially curved structural members. Each structural member is formed from two segments, each segment having a first end and a second end. The first end of one segment connects to the first end of the other segment, thereby forming the structural member. The second end of each segment connects to another segment of a different structural member, such that the structural members are oppositely and alternately positioned in a longitudinal direction, thereby forming the portion of the generally cylindrical structure. The portion expands from a compressed state to an expanded state by a spiraling motion of each of the structural members about the central longitudinal axis. Each structural member includes a crossbar connected to each of the two segments, thereby forming a closed cell region within each structural member. A ratio of a circumferential length of each structural member to a spacing between adjacent structural members is in the range of from about 1.8 to about 3.2.

According to another embodiment, the stent has a generally cylindrical structure with a central longitudinal axis, and at least a portion of the generally cylindrical structure is formed from an arrangement of circumferentially curved structural members. Each structural member is formed from two segments, each segment having a first end and a second end. The first end of one segment connects to the first end of the other segment, thereby forming the structural member. The second end of each segment connects to another segment of a different structural member, such that the structural members are oppositely and alternately positioned in a longitudinal direction, thereby forming the portion of the generally cylindrical structure. The portion expands from a compressed state to an expanded state by a spiraling motion of each of the structural members about the central longitudinal axis. Each structural member includes a crossbar connected to each of the two segments, thereby forming a closed cell region within each structural member. A ratio of a circumferential length of each structural member to a circumferential length of each closed cell region is in the range of from about 2.5 to about 6.5.

According to another embodiment, the stent has a generally cylindrical structure with a central longitudinal axis, and at least a portion of the generally cylindrical structure is formed from an arrangement of circumferentially curved structural members. Each structural member is formed from two segments, each segment having a first end and a second end. The first end of one segment connects to the first end of the other segment, thereby forming the structural member. The second end of each segment connects to another segment of a different structural member, such that the structural members are oppositely and alternately positioned in a longitudinal direction, thereby forming the portion of the generally cylindrical structure. The portion expands from a compressed state to an expanded state by a spiraling motion of each of the structural members about the central longitudinal axis. Each structural member includes at least one of: a nesting configuration, wherein the structural member nests within an open region formed by the segments in the compressed state, the portion of the generally cylindrical structure thereby having no overlapping regions; and a crossbar connected to each of the two segments, thereby forming a closed cell region within each structural member; and at least one of: a ratio of a circumferential length of each structural member to a spacing between adjacent structural members in the range of from about 1.8 to about 3.2; and a ratio of a circumferential length of each structural member to a circumferential length of each closed cell region in the range of from about 2.5 to about 6.5.

These and other features, aspects, and advantages will become better understood with regard to the following detailed description, appended claims and accompanying drawings.

### BRIEF DESCRIPTION OF THE DRAWINGS

The invention may be more fully understood by reading the following description in view of the drawings, in which:

FIG. 1 is a perspective view of a portion of a stent, showing a first embodiment of a generally cylindrical structure in an expanded state;

FIG. 2 is a planar view of four alternately and oppositely positioned structural members according to the first embodiment, showing the structural members in an uncurved configuration;

FIG. 3 is a perspective view of a portion of a stent, showing a second embodiment of a generally cylindrical structure in an expanded state;

FIG. 4 is a planar view of four alternately and oppositely positioned structural members according to the second embodiment, showing the structural members in an uncurved configuration;

FIG. 5 is a perspective view of a portion of a stent, showing a third embodiment of a generally cylindrical structure in an expanded state;

FIG. 6 is a planar view of four alternately and oppositely positioned structural members according to the third embodiment, showing the structural members in an uncurved configuration;

FIG. 7 is a perspective view of a portion of a stent, showing a fourth embodiment of a generally cylindrical structure in an expanded state;

FIG. 8 is a planar view of four alternately and oppositely positioned structural members according to the fourth embodiment, showing the structural members in an uncurved configuration;

FIG. 9 shows one embodiment of a portion of a generally cylindrical structure in a compressed, or crimped, state;

FIG. 10 shows the relationship between radial force per length and stent diameter (during crimping), as determined by finite element analysis (FEA), for several embodiments of the present stent and a prior art stent;

FIG. 11 shows the relationship between radial force and stent diameter, as determined by FEA, for stents of different thicknesses.

### DETAILED DESCRIPTION

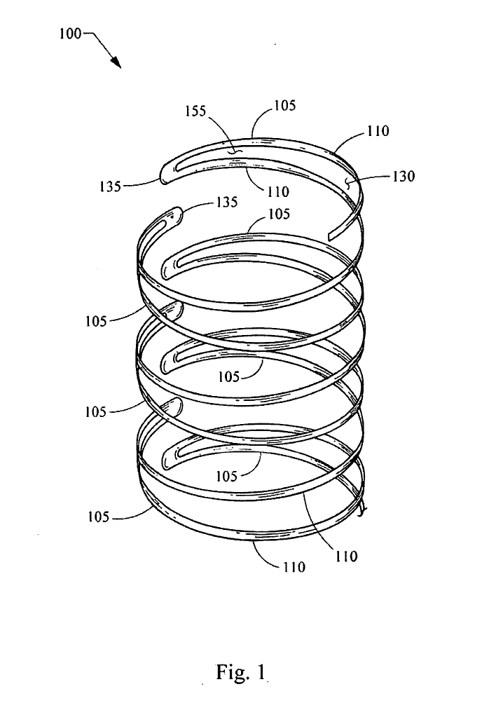

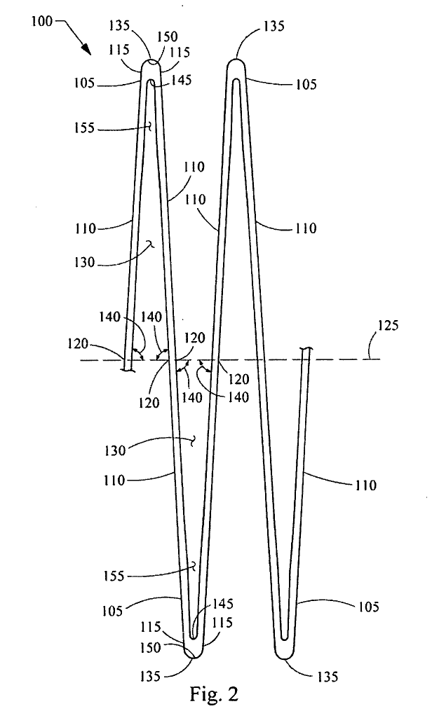

Referring now to the drawings, FIG. 1 shows a portion 100 of a generally cylindrical structure of a stent in an expanded state according to a first embodiment. FIG. 2 shows a planar view of four structural members 105 of the portion 100 of the generally cylindrical structure according to the first embodiment. In FIG. 2, the structural members 105 are shown oppositely and alternately positioned in an uncurved configuration.

As shown in FIG. 2, each structural member 105 is formed from two segments 110 with a first end 115 and a second end 120. The first end 115 of one segment 110 connects to the first end 115 of another segment 110, thereby forming the structural member 105. The second end 120 of each segment 110 connects to another segment 110 of a different structural member 105. Thus, the structural members 105 are oppositely and alternately positioned in a longitudinal direction. Preferably, the spacing and arrangement of the structural members 105 are designed to uniformly distribute the radial forces exerted on the vessel wall in the expanded configuration.

Each of the segments 110 forming the structural members 105 may have a width of 0.381 mm (0.015 inch). The thickness of each segment 110 may be 0.203 mm (0.008 inch) or have another value, such as 0.127 mm (0.005 inch) or 0.076 mm (0.003 inch). The total width of each structural member 105 as measured at the centerline 125 may be 2.95 mm (0.116 inch). The perpendicular distance from the centerline 125 to the tip 135 of each structural member 105 may be 16.5 mm (0.649 inch). This distance, from the centerline 125 to the tip 135, represents the circumferential length of the structural member. Each segment 110 extends from the centerline at an angle 140 of about +/−86.5 degrees and connects with the other segment 110 at the tip 135 of the structural member 105, creating an open region 130. The tip 135 may include a radius 150 of 0.508 mm (0.020 inch) and the open region 130 may include a radius 145 of 0.508 mm (0.020 inch).

According to this embodiment, the structural members 105 may have a spacing of 5.89 mm (0.232 inch), as measured from the tip 135 of one structural member 105 to the tip 135 of an adjacent structural member 105. The spacing and arrangement of the structural members 105 are designed to uniformly distribute the radial forces exerted on the vessel wall in the expanded configuration. A ratio of the circumferential length of each structural member 105 to the spacing of adjacent structural members 105 is equivalent to about 0.649/0.232, or about 2.8, according to this embodiment. Alternatively, the ratio of the circumferential length of each structural member 105 to the spacing of adjacent structural members 105 may be in the range of from about 1.8 to about 3.2. Preferably, the ratio may be in the range of from about 2.0 to about 3.0.

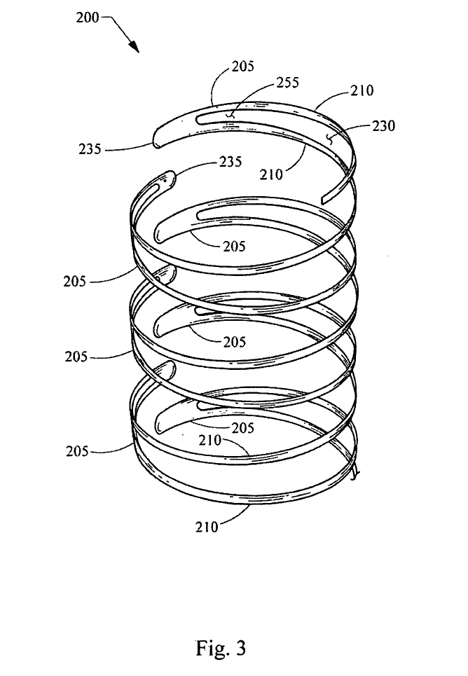

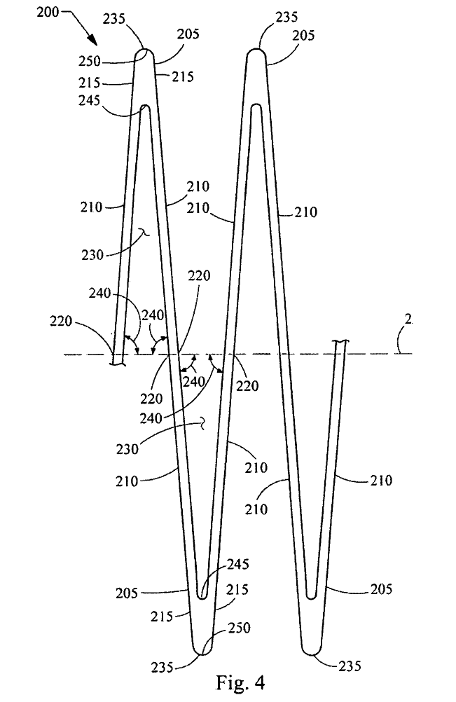

Referring again to the drawings, FIG. 3 shows a portion 200 of a generally cylindrical structure of a stent in an expanded state according to a second embodiment. FIG. 4 shows a planar view of four structural members 205 of the portion 200 of the generally cylindrical structure according to the second embodiment. In FIG. 4, the structural members 205 are shown oppositely and alternately positioned in an uncurved configuration.

As shown in FIG. 4, each structural member 205 is formed from two segments 210 with a first end 215 and a second end 220. The first end 215 of one segment 210 connects to the first end 215 of another segment 210, thereby forming the structural member 205. The second end 220 of each segment 210 connects to another segment 210 of a different structural member 205. Thus, the structural members 205 are oppositely and alternately positioned along the longitudinal direction. Preferably, the spacing and arrangement of the structural members 205 are designed to uniformly distribute the radial forces exerted on the vessel wall in the expanded configuration.

Each of the segments 210 forming the structural members 205 may have a width of 0.508 mm (0.020 inch). The thickness of each segment 210 may be 0.203 mm (0.008 inch) or have another value, such as 0.127 mm (0.005 inch) or 0.076 mm (0.003 inch). The total width of each structural member 205 as measured at the centerline 225 may be 3.61 mm (0.142 inch). The perpendicular distance from the centerline 225 to the tip 235 of each structural member 205 may be 16.5 mm (0.649 inch). This distance, from the centerline 225 to the tip 235, represents the circumferential length of the structural member. Each segment 210 extends from the centerline at an angle 240 of about +/−85.4 degrees and connects with the other segment 210 at the tip 235 of the structural member 205, creating an open region 230. The tip 235 may include a radius 250 of 0.508 mm (0.020 inch) and the open region 230 may include a radius 245 of 0.508 mm (0.020 inch).

According to this embodiment, the structural members 205 may have a spacing of 7.21 mm (0.284 inch), as measured from the tip 235 of one structural member 205 to the tip 235 of an adjacent structural member 205. The spacing and arrangement of the structural members 205 are designed to uniformly distribute the radial forces exerted on-the vessel wall in the expanded configuration. A ratio of the circumferential length of each structural member 205 to the spacing of adjacent structural members 205 is equivalent to about 0.649/0.284 or about 2.3, according to this embodiment. Alternatively, the ratio of the circumferential length of each structural member 205 to the spacing of adjacent structural members 205 may be in the range of from about 1.8 to about 3.2. Preferably, the ratio may be in the range of from about 2.0 to about 3.0.

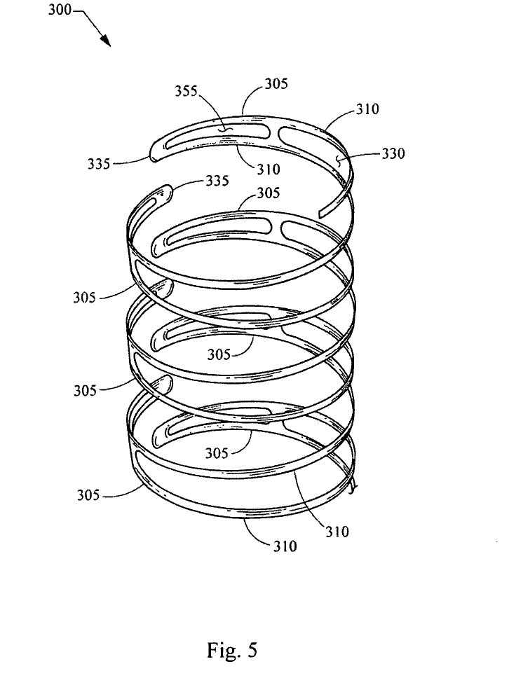

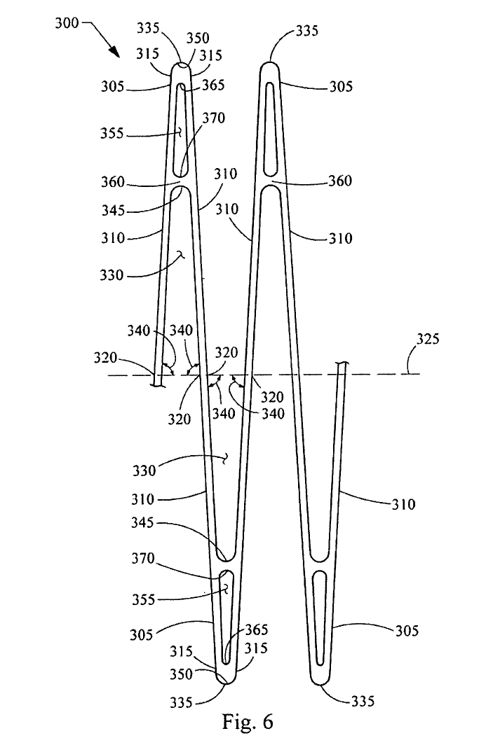

Referring again to the drawings, FIG. 5 shows a portion 300 of a generally cylindrical structure of a stent in an expanded state according to a third embodiment. FIG. 6 shows a planar view of four structural members 305 of the portion 300 of the generally cylindrical structure according to the third embodiment. In FIG. 6, the structural members 305 are shown oppositely and alternately positioned in an uncurved configuration.

As shown in FIG. 6, each structural member 305 is formed from two segments 310 with a first end 315 and a second end 320. The first end 315 of one segment 310 connects to the first end 315 of another segment 310, thereby forming the structural member 305. The second end 320 of each segment 310 connects to another segment 310 of a different structural member 305. Thus, the structural members 305 are oppositely and alternately positioned in a longitudinal direction. Preferably, the spacing and arrangement of the structural members 305 are designed to uniformly distribute the radial forces exerted on the vessel wall in the expanded configuration.

FIG. 6 also shows a closed cell region 355. The closed cell region 355 is formed by a crossbar 360 connecting the two segments 310 included within each structural member 305. The inclusion of a closed cell region 355 preferably improves the circumferential uniformity of the stiffness of the structural member 305 in the compressed or expanded state.

Each of the segments 310 forming the structural members 305 may have a width of 0.381 mm (0.015 inch). The thickness of each segment 310 may be 0.203 mm (0.008 inch) or have another value, such as 0.127 mm (0.005 inch) or 0.076 mm (0.003 inch). The total width of each structural member 305 as measured at the centerline 325 may be 2.95 mm (0.116 inch). The perpendicular distance from the centerline 325 to the tip 335 of each structural member 305 may be 16.5 mm (0.649 inch). This distance, from the centerline 325 to the tip 335, represents the circumferential length of the structural member. Each segment 310 extends from the centerline at an angle 340 of about +/−86.5 degrees and connects with the other segment 310 at the end of an open region 330 and at the tip 335 of the structural member 305. The perpendicular distance from the centerline 325 to the end of the open region 330 may be 10.0 mm (0.395 inch). The end of the open region 330 may include a radius 345 of 0.508 mm (0.020 inch), and the tip 335 may include a radius 350 of 0.508 mm (0.020 inch). In this embodiment, each structural member 305 includes a closed cell region 355. The closed cell region 355 may have a circumferential length of 5.00 mm (0.197 inch), wherein the circumferential length represents the total length of the closed cell region 355. The closed cell region 355 may include a first radius 365 of 0.178 mm (0.007 inch) and a second radius 370 of 0.381 mm (0.015 inch). The closed cell region 355 may begin at a distance of 10.4 mm (0.410 inch) from the centerline 325. The ratio of the circumferential length of each structural member 305 to the circumferential length of each closed cell region 355 according to this embodiment is equivalent to about 0.649/0.197, or about 3.3. Alternatively, the ratio of the circumferential length of each structural member 305 to the circumferential length of each closed cell region 355 may be in the range of from about 2.5 to about 6.5. Preferably, the ratio may be in the range of from about 3.0 to about 6.0.

According to this embodiment, the structural members 305 may have a spacing of 5.89 mm (0.232 inch), as measured from the tip 335 of one structural member 305 to the tip 335 of an adjacent structural member 305. The spacing and arrangement of the structural members 305 are designed to uniformly distribute the radial forces exerted on the vessel wall in the expanded configuration. A ratio of the circumferential length of each structural member 305 to the spacing of adjacent structural members 305 is equivalent to about 0.649/0.232 or about 2.8, according to this embodiment. Alternatively, the ratio of the circumferential length of each structural member 305 to the spacing of adjacent structural members 305 may be in the range of from about 1.8 to about 3.2. Preferably, the ratio may be in the range of from about 2.0 to about 3.0.

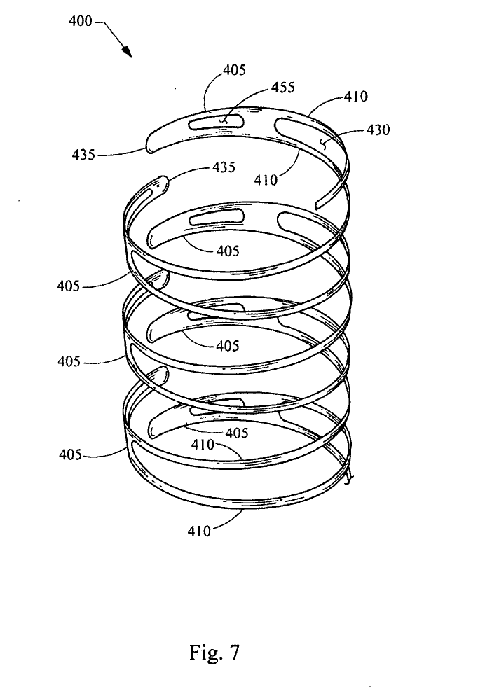

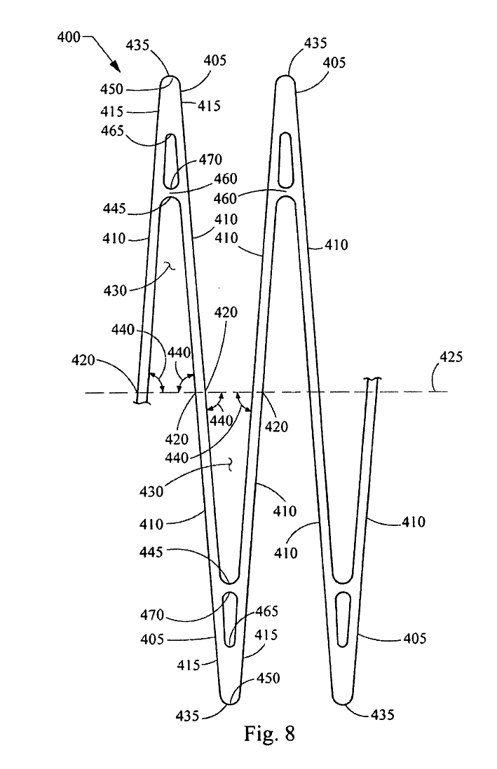

Referring again to the drawings, FIG. 7 shows a portion 400 of a generally cylindrical structure of a stent in an expanded state according to a fourth embodiment. FIG. 8 shows a planar view of four structural members 405 of the portion 400 of the generally cylindrical structure according to the fourth embodiment. In FIG. 8, the structural members 405 are shown oppositely and alternately positioned in an uncurved configuration.

As shown in FIG. 8, each structural member 405 is formed from two segments 410 with a first end 415 and a second end 420. The first end 415 of one segment 410 connects to the first end 415 of another segment 410, thereby forming the structural member 405. The second end 420 of each segment 410 connects to another segment 410 of a different structural member 405. Thus, the structural members 405 are oppositely and alternately positioned along the longitudinal direction. Preferably, the spacing and arrangement of the structural members 405 are designed to uniformly distribute the radial forces exerted on the vessel wall in the expanded configuration.

FIG. 8 also shows a closed cell region 455. The closed cell region 455 is formed by a crossbar 460 connecting the two segments 410 included within each structural member 405. The inclusion of a closed cell region 455 preferably improves the circumferential uniformity of the stiffness of the structural member 405 in the compressed or expanded state.

Each of the segments 410 forming the structural members 405 may have a width of 0.508 mm (0.020 inch). The thickness of each segment 410 may be 0.008 inch or have another value, such as 0.127 mm (0.005 inch) or 0.076 mm (0.003 inch). The total width of each structural member 405 as measured at the centerline 425 may be 3.61 mm (0.142 inch). The perpendicular distance from the centerline 425 to the tip 435 of each structural member 405 may be 16.5 mm (0.649 inch). This distance, from the centerline 425 to the tip 435, represents the circumferential length of the structural member. Each segment 410 extends from the centerline at an angle 440 of about +/−85.4 degrees and connects with the other segment 410 at the end of an open region 430 and at the tip 435 of the structural member 405. The perpendicular distance from the centerline 425 to the end of the open region 430 may be 10.2 mm (0.400 inch). The end of the open region 430 may include a radius 445 of 0.508 mm (0.020 inch), and the tip 435 may include a radius 450 of 0.508 mm (0.020 inch). In this embodiment, each structural member 405 includes a closed cell region 455. The closed cell region 455 may have a circumferential length of 2.92 mm (0.115 inch), wherein the circumferential length represents the total length of the closed cell region 455. The closed cell region 455 may include a first radius 465 of 0.229 mm (0.009 inch) and a second radius 470 of 0.406 mm (0.016 inch). The closed cell region 455 may begin at a distance of 10.5 mm (0.414 inch) from the centerline 425. The ratio of the circumferential length of each structural member 405 to the circumferential length of each closed cell region 455 according to this embodiment is equivalent to about 0.649/0.115, or about 5.6. Alternatively, the ratio of the circumferential length of each structural member 205 to the circumferential length of each closed cell region 255 may be in the range of from about 2.5 to about 6.5. Preferably, the ratio may be in the range of from about 3.0 to about 6.0.

According to this embodiment, the structural members 405 may have a spacing of 7.21 mm (0.284 inch), as measured from the tip 435 of one structural member 405 to the tip 435 of an adjacent structural member 405. The spacing and arrangement of the structural members 405 are designed to uniformly distribute the radial forces exerted on the vessel wall in the expanded configuration. A ratio of the circumferential length of each structural member 405 to the spacing of adjacent structural members 405 is equivalent to about 0.649/0.284 or about 2.3, according to this embodiment. Alternatively, the ratio of the circumferential length of each structural member 405 to the spacing of adjacent structural members 405 may be in the range of from about 1.8 to about 3.2. Preferably, the ratio may be in the range of from about 2.0 to about 3.0.

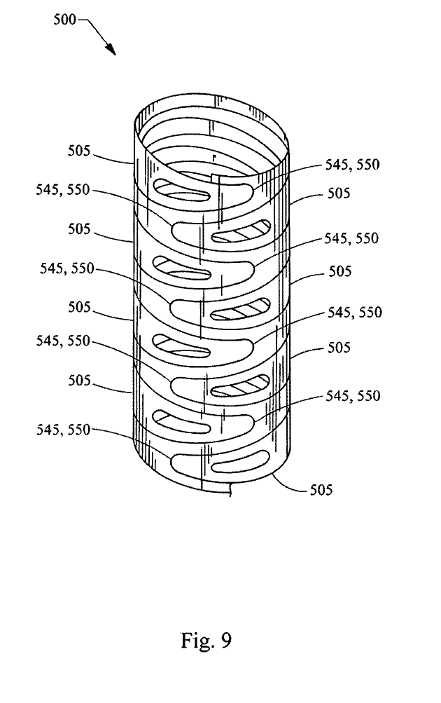

FIG. 9 shows a portion 500 of a generally cylindrical structure of a stent in a compressed, or crimped, state according to another embodiment. In the compressed state, each of the structural members 505 may nest within an open region formed by the segments that form the structural member 505. Thus, the portion 500 of the generally cylindrical structure has no overlapping regions. In the maximally crimped state, the radius 550 of the tip of each structural member may be adjacent to the radius 545 of the open region of the same structural member. The portion 500 of the generally cylindrical structure may thus resemble a solid tube.

The stents described above are preferably self-expandable and formed from a superelastic material. The term “superelastic material”, as used herein, refers to a material that exhibits a substantial amount of elastic (i.e., recoverable) deformation, or strain, in response to an applied stress. Typically, superelastic materials can achieve elastic strains of at least several percent. Upon removal of the applied stress, the elastic strain that was induced by the applied stress is recovered and the material returns to its original, undeformed configuration. One example of a superelastic material is Nitinol, which is a superelastic nickel-titanium alloy that can achieve an elastic strain of about 8%. In contrast, conventional metal alloys, such as 304 stainless steel, typically achieve elastic strains of only a fraction of a percent. Materials exhibiting superelastic behavior are sometimes referred to as shape memory materials or pseudoelastic materials.

Superelastic materials are particularly advantageous for self-expandable stents. Superelastic materials are also advantageous for stents implanted into the SFA and other peripheral arteries. The preferred superelastic material used in the described stents includes nickel and titanium. In one embodiment, the superelastic material is Nitinol. The superelastic material may also include a ternary element, a quaternary element and/or additional elements.

The described stents may be deployed in a vessel within the body using standard deployment techniques known to medical professionals. For example, the stent may be mounted within a retaining sheath which contacts the outer surface of the stent and retains the stent in a compressed state for delivery into a vessel. A hollow needle may be used to penetrate the vessel, and a guide wire may be threaded through the needle into the vessel. The needle may then be removed and replaced with an introduction catheter, which generally acts as a port through which intraluminal devices, including stents, may then be passed to gain access to a vessel. The compressed stent and the retaining sheath may then be passed through the introduction catheter into the vessel. Once the stent is positioned within the vessel adjacent to the site to be treated, the retaining sheath may be retracted, thereby causing the stent to expand from the compressed state to an expanded state. In the expanded state, the stent contacts and exerts a radial force on the vessel wall. The retaining sheath and the introduction catheter may then be withdrawn from the vessel. According to one embodiment, the present stent may be advantageously deployed in a vessel having an inner diameter of from about 7.5 mm to about 9 mm. The stent also may be used in vessels of other diameters.

Standard laser cutting techniques which are known in the art may be employed to fabricate stents described above. For example, the stent structure may be fabricated by laser cutting the structural members from a tube. As a result, each segment of the structural members has a generally curved inner diameter and outer diameter and generally flat side surfaces.

### EXAMPLE 1

A finite element analysis (FEA) was carried out to calculate radial force for several embodiments of the present stent. These results were compared to FEA data for a prior art stent, the Cook Zilver®. In the FEA, the stents underwent a basic crimp from a 10 mm outer diameter to a 7.5 mm outer diameter while total radial force was calculated.

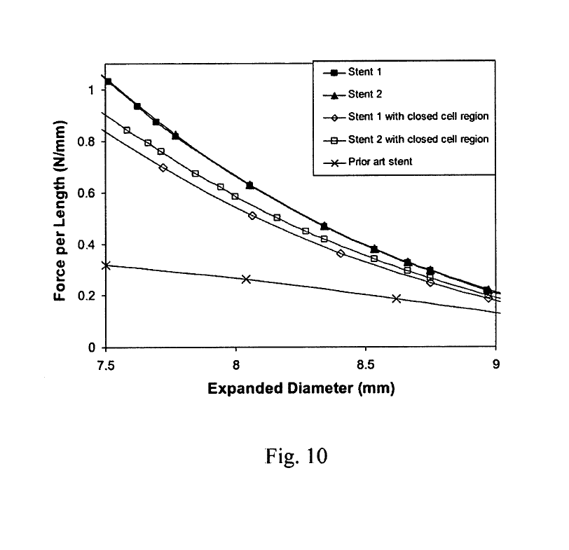

The radial force data were then normalized by the length of each stent. The results, in terms of radial force per length versus expanded diameter, are shown graphically in FIG. 10 for the following stents: a prior art stent (Zilver®, 10 mm in length); Stent 1 (10.67 mm in length), which corresponds to the first embodiment of the inventive stent as shown in FIG. 1 and FIG. 2; Stent 2 (12.84 mm in length), which corresponds to the second embodiment of the inventive stent as shown in FIG. 3 and FIG. 4; Stent 1 including a closed cell region (10.67 mm in length), which corresponds to the third embodiment of the inventive stent as shown in FIG. 5 and FIG. 6; and Stent 2 including a closed cell region (12.84 mm in length), which corresponds to the fourth embodiment of the inventive stent as shown in FIG. 7 and FIG. 8. The FEA results show that stents designed according to the present invention exhibit a higher radial force per length in comparison with the prior art stent. At an expanded diameter of 7.5 mm, the radial force exerted by the inventive stents is approximately 150% to 220% higher than that of the prior art stent.

### EXAMPLE 2

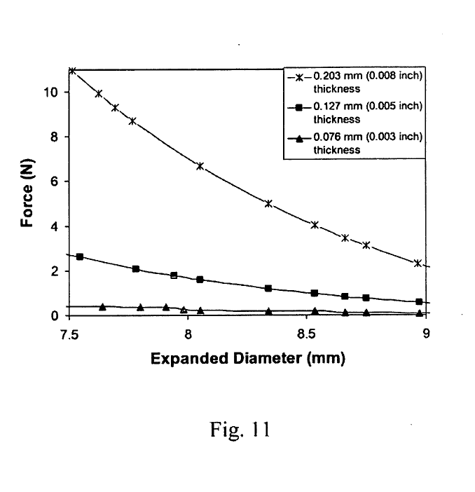

FIG. 11 shows the relationship between radial force and stent thickness for the first embodiment of the inventive stent. As in the preceding example, the stents underwent a basic crimp from a 10 mm outer diameter to a 7.5 mm outer diameter while total radial force was calculated using FEA.

The FEA data presented graphically in FIG. 11 show radial force versus expanded diameter for three different stent thicknesses: 0.203 mm (0.008 inch), 0.127 mm (0.005 inch), and 0.076 mm (0.003 inch). FIG. 11 shows that radial force is highest for the largest stent thickness (0.203 mm (0.008 inch)).

The stents described herein may be advantageously used in the superficial femoral artery (SFA), which may experience large and repetitive external traumas. Such stents also may be useful in other applications. When used to treat the SFA and other peripheral arteries, the described stents may provide desirable properties to successfully treat occlusions and other conditions. For example, the described stents may provide a substantial amount of elastic (i.e., recoverable) deformation in response to external traumas, a high and uniform radial force when deployed, high axial flexibility in bending and torsion, a long fatigue life, and a uniform circumferential stiffness.

Although the present invention has been described in considerable detail with reference to certain embodiments thereof, other embodiments are possible without departing from the present invention. The spirit and scope of the appended claims should not be limited, therefore, to the description of the preferred embodiments contained herein. All devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

We claim:

1. An expandable stent for medical implantation, comprising:
a generally cylindrical structure having a central longitudinal axis, wherein at least a portion of the generally cylindrical structure is formed from an arrangement of circumferentially curved structural members;
wherein each structural member comprises two segments, each segment comprising a first end and a second end, the first end of one segment being connected to the first end of the other segment, thereby forming the structural member, and the second end of each segment being connected to another segment of a different structural member, such that the structural members are oppositely and alternately positioned along a longitudinal direction, thereby forming the portion of the generally cylindrical structure, the portion expanding from a compressed state to an expanded state by a spiraling motion of each of the structural members about the central longitudinal axis; and
wherein each of the structural members nests within an open region formed by the segments in the compressed state, the portion of the generally cylindrical structure thereby having no overlapping regions; and
wherein a ratio of a circumferential length of each structural member to a spacing between adjacent structural members is in the range of from about 1.8 to about 3.2.

2. The expandable stent according to claim 1, wherein the ratio of the circumferential length of each structural member to the spacing between adjacent structural members is in the range of from about 2.0 to about 3.0.

3. The expandable stent according to claim 1, wherein the generally cylindrical structure is self-expandable and formed from a superelastic material.

4. The expandable stent according to claim 1, wherein the portion of the generally cylindrical structure is fabricated by laser cutting the structural members from a tube, each of the segments thereby comprising a generally curved inner and outer diameter and generally flat side surfaces.

5. The expandable stent according to claim 1, wherein each structural member comprises a crossbar connected to each of the two segments, thereby forming a closed cell region within each structural member.

6. The expandable stent according to claim 5, wherein a ratio of a circumferential length of each structural member to a circumferential length of each closed cell region is in the range of from about 2.5 to about 6.5.

7. The expandable stent according to claim 1,
wherein the generally cylindrical structure is self-expandable and formed from a superelastic material;
wherein the portion of the generally cylindrical structure is fabricated by laser cutting the structural members from a tube, each of the segments thereby comprising a generally curved inner and outer diameter and generally flat side surfaces;
wherein each structural member comprises a crossbar connected to each of the two segments, thereby forming a closed cell region within each structural member; and
wherein a ratio of a circumferential length of each structural member to a circumferential length of each closed cell region is in the range of from about 2.5 to about 6.5.

8. An expandable stent for medical implantation, comprising:
a generally cylindrical structure having a central longitudinal axis, wherein at least a portion of the generally cylindrical structure is formed from an arrangement of circumferentially curved structural members;
wherein each structural member comprises two segments, each segment comprising a first end and a second end, the first end of one segment being connected to the first end of the other segment, thereby forming the structural member, and the second end of each segment being connected to another segment of a different structural member, such that the structural members are oppositely and alternately positioned along a longitudinal direction, thereby forming the portion of the generally cylindrical structure, the portion expanding from a compressed state to an expanded state by a spiraling motion of each of the structural members about the central longitudinal axis;
wherein each structural member comprises a crossbar connected to each of the two segments, thereby forming a closed cell region within each structural member; and
wherein a ratio of a circumferential length of each structural member to a spacing between adjacent structural members is in the range of from about 1.8 to about 3.2.

9. The expandable stent according to claim 8, wherein the ratio of the circumferential length of each structural member to the spacing between adjacent structural members is in the range of from about 2.0 to about 3.0.

10. The expandable stent according to claim 8, wherein each of the structural members nests within an open region formed by the segments in the compressed state, the portion of the generally cylindrical structure thereby having no overlapping regions.

11. The expandable stent according to claim 8, wherein a ratio of a circumferential length of each structural member to a circumferential length of each closed cell region is in the range of from about 2.5 to about 6.5.

12. The expandable stent according to claim 8, wherein the generally cylindrical structure is self-expandable and is formed from a superelastic material.

13. The expandable stent according to claim 8, wherein the portion of the generally cylindrical structure is fabricated by laser cutting the structural members from a tube, each of the segments thereby comprising a generally curved inner and outer diameter and generally flat side surfaces.

14. The expandable stent according to claim 8,
wherein each of the structural members nests within an open region formed by the segments in the compressed state, the portion of the generally cylindrical structure thereby having no overlapping regions;
wherein a ratio of a circumferential length of each structural member to a circumferential length of each closed cell region is in the range of from about 2.5 to about 6.5;
wherein the generally cylindrical structure is self-expandable and is formed from a superelastic material; and
wherein the portion of the generally cylindrical structure is fabricated by laser cutting the structural members from a tube, each of the segments thereby comprising a generally curved inner and outer diameter and generally flat side surfaces.

15. An expandable stent for medical implantation, comprising:
a generally cylindrical structure having a central longitudinal axis, wherein at least a portion of the generally cylindrical structure is formed from an arrangement of circumferentially curved structural members;
wherein each structural member comprises two segments, each segment comprising a first end and a second end, the first end of one segment being connected to the first end of the other segment, thereby forming the structural member, and the second end of each segment being connected to another segment of a different structural member, such that the structural members are oppositely and alternately positioned along a longitudinal direction, thereby forming the portion of the generally cylindrical structure, the portion expanding from a compressed state to an expanded state by a spiraling motion of each of the structural members about the central longitudinal axis;
wherein each structural member comprises a crossbar connected to each of the two segments, thereby forming a closed cell region within each structural member; and
wherein a ratio of a circumferential length of each structural member to a circumferential length of each closed cell region is in the range of from about 2.5 to about 6.5.

16. The expandable stent according to claim 15, wherein the ratio of the circumferential length of each structural member to the circumferential length of each closed cell region is in the range of from about 3.0 to about 6.0.

17. The expandable stent according to claim 15, wherein a ratio of a circumferential length of each structural member to a spacing between adjacent structural members is in the range of from about 1.8 to about 3.2.

18. The expandable stent according to claim 15, wherein each of the structural members nests within an open region formed by the segments in the compressed state, the portion of the generally cylindrical structure thereby having no overlapping regions.

19. The expandable stent according to claim 15, wherein the portion of the generally cylindrical structure is fabricated by laser cutting the structural members from a tube, each of the segments thereby comprising a generally curved inner and outer diameter and generally flat side surfaces.

20. The expandable stent according to claim 15, wherein the generally cylindrical structure is self-expandable and is formed from a superelastic material.

21. The expandable stent according to claim 15,
wherein a ratio of a circumferential length of each structural member to a spacing between adjacent structural members is in the range of from about 1.8 to about 3.2;
wherein each of the structural members nests within an open region formed by the segments in the compressed state, the portion of the generally cylindrical structure thereby having no overlapping regions;
wherein the portion of the generally cylindrical structure is fabricated by laser cutting the structural members from a tube, each of the segments thereby comprising a generally curved inner and outer diameter and generally flat side surfaces; and
wherein the generally cylindrical structure is self-expandable and is formed from a superelastic material.

22. An expandable stent for medical implantation, comprising:
a generally cylindrical structure having a central longitudinal axis, wherein at least a portion of the generally cylindrical structure is formed from an arrangement of circumferentially curved structural members;
wherein each structural member comprises two segments, each segment comprising a first end and a second end, the first end of one segment being connected to the first end of the other segment, thereby forming the structural member, and the second end of each segment being connected to another segment of a different structural member, such that the structural members are oppositely and alternately positioned along a longitudinal direction, thereby forming the portion of the generally cylindrical structure, the portion expanding from a compressed state to an expanded state by a spiraling motion of each of the structural members about the central longitudinal axis;
wherein each structural member further comprises at least one of:
a nesting configuration, wherein the structural member nests within an open region formed by the segments in the compressed state, the portion of the generally cylindrical structure thereby having no overlapping regions, and
a crossbar connected to each of the two segments, thereby forming a closed cell region within each structural member; and
at least one of:
a ratio of a circumferential length of each structural member to a spacing between adjacent structural members in the range of from about 1.8 to about 3.2, and
a ratio of a circumferential length of each structural member to a circumferential length of each closed cell region in the range of from about 2.5 to about 6.5.

23. The expandable stent according to claim 22, wherein the portion of the generally cylindrical structure is fabricated by laser cutting the structural members from a tube, each of the segments thereby comprising a generally curved inner and outer diameter and generally flat side surfaces.

24. The expandable stent according to claim 22, wherein the generally cylindrical structure is self-expandable and is formed from a superelastic material.
